(19) United States
(12) Patent Application Publication
(10) Pub. No.: US 2011/0319611 A1
(43) Pub. Date: Dec. 29, 2011

(54) BENZOXAZINONE COMPOUND, METHOD FOR PRODUCING THE SAME, AND PHARMACEUTICAL COMPOSITION CONTAINING THE SAME

(75) Inventors: Pei-Wen Hsieh, Kwei-Shan (TW); Tsong-Long Hwang, Kwei-Shan (TW); Huang-Ping Yu, Kwei-Shan (TW)

(73) Assignee: Chang Gung University, Kwei-Shan (TW)

(21) Appl. No.: 12/967,781

(22) Filed: Dec. 14, 2010

(30) Foreign Application Priority Data
Jun. 24, 2010 (TW) 099120609

(51) Int. Cl. C07D 265/22

(52) U.S. Cl. 544/92

(57) ABSTRACT

A benzoxazinone compound having the following formula (I):

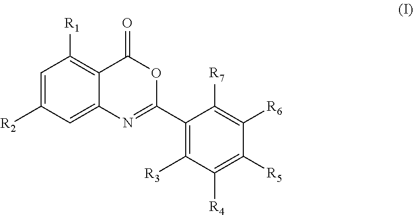

wherein
one of R₁ and R₂ is H, and the other of R₁ and R₂ is a halogen group, and R₁ and R₂ cannot be the same at the same time; and
R₃, R₄, R₅, R₆, and R₇ are independently selected from the group consisting of H, a halogen group, a C₁-C₄ alkyl group, and a C₁-C₄ alkoxyl group.

### CROSS-REFERENCE TO RELATED APPLICATION

This application claims priority of Taiwanese Application No. 099120609, filed on Jun. 24, 2010.

### BACKGROUND OF THE INVENTION

1. Field of the Invention

This invention relates to a benzoxazinone compound, a method for preparing the same, and a pharmaceutical composition containing the same.

2. Description of the Related Art

Neutrophils play a vital role in the defense of the human body against infections. In response to inflammatory stimulus, activated neutrophils secrete a series of cytotoxins, such as superoxide anion (O₂^{•-}), precursors of other reactive oxygen species, granule proteases, and bioactive lipids. Inappropriate neutrophil responses can result in inflammatory disorders and tissue destruction. The inflammatory disorders attributed to the inappropriate neutrophil response include lung injury, chronic obstructive pulmonary disease, acute respiratory distress syndrome, cystic fibrosis, ischemic-reperfusion injury, glomerulonephritis, arthritis, bullous pemphigoid, and sepsis.

Neutrophil elastase (NE), also referred to as a leukocyte elastase or lysosomal elastase, is a kind of granule protease. NE is a 30-kDa glycoprotein chymotrypsin-like serine proteinase and is stored in azurophil granules of the neutrophil. NE is a major secreted product of activated neutrophils and a major contributor to destruction of tissue in chronic inflammatory diseases. Therefore, inhibition of activity of the neutrophil elastase can be deemed an important therapy for inflammatory disorders.

Substituted benzoxazinone compounds have been proven to exhibit inhibition of NE activity. In particular, the substituted benzoxazinone compounds can be reacted with active site serine 195 of neutrophil elastase to form an acyl enzyme intermediate, thereby resulting in inhibition of NE activity.

A. Krantz et al. disclosed a 2-phenyl-4H-3,1-benzoxazin-4-one compound having the following formula, which exhibits inhibiting effect on NE activity (see A. Krantz et al., J. Med. Chem., 33:464-479, 1990):

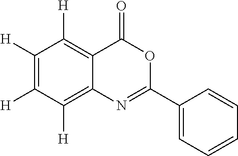

The applicants of the present invention have synthesized a series of 2,8-substituted benzoxazinones having the following general formula.

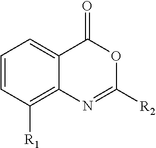

The effects of the series of the 2,8-substituted benzoxazinones on inhibition of NE release and O₂^{•-} generation were investigated. The experiments reveal that only some of benzoxazinones exhibit the desired activities (see P. W. Hsieh et al., Bioorg. Med. Chem. Lett., 15:2786-2789, 2005).

Although a series of substituted benzoxazinones exhibiting NE inhibition have been provided, there is still a need in the art to provide a compound having a superior effect on inhibition of NE activity.

### SUMMARY OF THE INVENTION

Therefore, the object of the present invention is to provide a benzoxazinone compound having a superior effect on inhibition of NE activity.

According to one aspect of this invention, there is provided a benzoxazinone compound having the following formula (I):

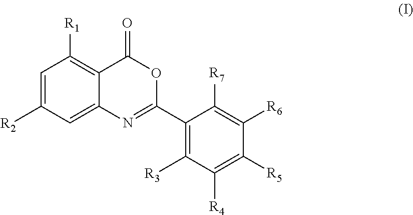

wherein

one of R₁ and R₂ is H, and the other of R₁ and R₂ is a halogen group; and

R₃, R₄, R₅, R₆, and R₇ are independently selected from the group consisting of H, a halogen group, a C₁-C₄ alkyl group, and a C₁-C₄ alkoxyl group.

According to another aspect of this invention, there is provided a pharmaceutical composition for treatment of an inflammatory disorder, including the benzoxazinone compound of formula (I) or the pharmaceutically acceptable salt thereof.

According to yet another aspect of this invention, there is provided a method for producing the benzoxazinone compound of formula (I), including:

reacting a compound of formula (II):

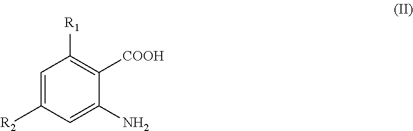

with a compound of formula (III):

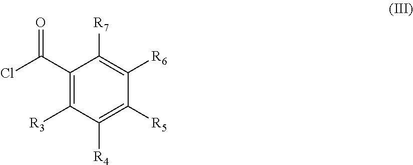

wherein R₁, R₂, R₃, R₄, R₅, R₆, and R₇ have the same definitions as R₁, R₂, R₃, R₄, R₅, R₆, and R₇ in formula (I), respectively.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is a plot illustrating the effect of a benzoxazinone compound E7 on water content in lung tissue, in which experimental groups 1 and 2 were subjected to a trauma-hemorrhage procedure, and were treated with a vehicle and the benzoxazinone compound E7, respectively, and in which sham-operated groups 1 and 2 were not subjected to a trauma-hemorrhage procedure, and were treated with a vehicle and the benzoxazinone compound E7, respectively, “***” being indicative of p<0.05 when the experimental groups 1 and 2 were compared with the sham-operated groups 1 and 2, respectively, “###” being indicative of p<0.001 when the experimental group 1 was compared with the experimental group 2; and

FIG. 2 is a plot illustrating the effect of the benzoxazinone compound E7 on inhibition of MPO activity, in which the experimental groups 1 and 2 were subjected to a trauma-hemorrhage procedure, and were treated with a vehicle and the benzoxazinone compound E7, respectively, and in which the sham-operated groups 1 and 2 were not subjected to a trauma-hemorrhage procedure, and were treated with a vehicle and the benzoxazinone compound E7, respectively, “***” being indicative of p<0.05 when the experimental groups 1 and 2 were compared with the sham-operated groups 1 and 2, respectively, “##” being indicative of p<0.01 when the experimental group 1 was compared with the experimental group 2.

### DETAILED DESCRIPTION OF THE PREFERRED EMBODIMENTS

A benzoxazinone compound of the present invention has the following formula (I):

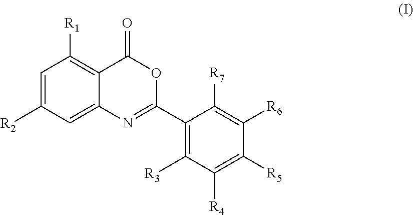

wherein

one of R₁ and R₂ is H, and the other of R₁ and R₂ is a halogen group; and

R₃, R₄, R₅, R₆, and R₇ are independently selected from the group consisting of H, a halogen group, a C₁-C₄ alkyl group, and a C₁-C₄ alkoxyl group.

The term “C₁-C₄ alkyl group” referred to herein is a straight or branched saturated monovalent hydrocarbon group having 1 to 4 carbon atoms. Examples of the C₁-C₄ alkyl group include, but are not limited to, methyl, ethyl, propyl, isopropyl, n-butyl, iso-butyl, sec-butyl, tert-butyl, and branched chain isomers thereof.

The term “C₁-C₄ alkoxyl group” referred to herein is a group having a moiety of —OR′, in which R′ is a C₁-C₄ alkyl group as defined above. Examples of the C₁-C₄ alkoxyl group include, but are not limited to, methoxyl, ethoxyl, n-propoxyl, iso-propoxyl, n-butoxyl, iso-butoxyl, sec-butoxyl, and tert-butoxyl.

The benzoxazinone compound of this invention is preferably selected from:
7-chloro-2-(2′-fluorophenyl)-4H-benzo[d][1,3]oxazin-4-one,
7-chloro-2-(2′-chlorophenyl)-4H-benzo[d][1,3]oxazin-4-one,
7-chloro-2-(2′-bromophenyl)-4H-benzo[d][1,3]oxazin-4-one,
7-chloro-2-(2′-methoxylphenyl)-4H-benzo[d][1,3]oxazin-4-one,
5-chloro-2-(2′-fluorophenyl)-4H-benzo[d][1,3]oxazin-4-one,
5-chloro-2-(2′-chlorophenyl)-4H-benzo[d][1,3]oxazin-4-one,
5-chloro-2-(2′-bromophenyl)-4H-benzo[d][1,3]oxazin-4-one,
5-chloro-2-(2′-methylphenyl)-4H-benzo[d][1,3]oxazin-4-one, and
5-chloro-2-(2′-methoxylphenyl)-4H-benzo[d][1,3]oxazin-4-one.

The benzoxazinone compound of the present invention can exist in free form, or where appropriate, as a pharmaceutically acceptable sait, solvate or hydrate thereof.

The pharmaceutically acceptable salt of the benzoxazinone compound may be, but is not limited to, a salt with an inorganic acid, such as HCl, HBr, H₂SO₄, and H₂PO₄, a salt with an organic acid, such as acetate, maleate, tartrate, and methanesulfonate, and a salt with an amino acid, such as arginine, aspartic acid, and glutamic acid.

A method for producing the benzoxazinone compound of formula (I) includes:

reacting a compound of formula (II):

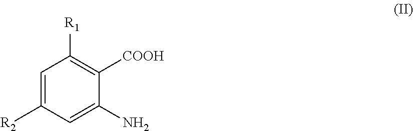

with a compound of formula (III):

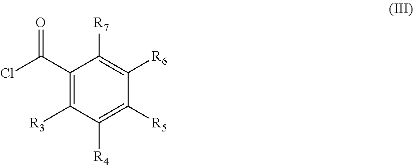

wherein R₁, R₂, R₃, R₄, R₅, R₆, and R₇ have the same definitions as R₁, R₂, R₃, R₄, R₅, R₆, and R₇ in formula (I), respectively.

A pharmaceutical composition capable of inhibiting activity of neutrophile elastase, includes the benzoxazinone compound of formula (I) or the pharmaceutically acceptable salt thereof.

The pharmaceutical composition is capable of inhibiting activity of neutrophile elastase, and can be used to treat inflammatory disorders, such as lung injury, chronic obstructive pulmonary disease, acute respiratory distress syndrome, cystic fibrosis, ischemic-reperfusion injury, glomerulonephritis, arthritis, bullous pemphigoid, and sepsis.

The pharmaceutical composition of this invention further includes a pharmaceutically acceptable carrier. The pharmaceutically acceptable carrier includes at least one of the following agents: a solvent, an emulsifier, a suspending agent, a decomposer, a binding agent, an excipient, a stabilizing agent, a chelating agent, a diluent, a gelling agent, a preservative, a lubricant, etc.

The pharmaceutical composition may be parenterally or orally administrable and can be formulated into a dosage form of e.g., sterile aqueous solutions, dispersions, sterile powders, tablets, troches, pills, capsules, etc.

This invention will be further described by way of the following examples. However, it should be understood that the following examples are solely for the purpose of illustration and should not be construed as limiting the invention in practice.

### EXAMPLES

### General Procedure

Thin layer chromatography (TLC) was conducted using a pre-coated silica gel 60 F₂₅₄ plate, commercially available from Merck & Co., Inc., and was detected at 254 nm.

¹H-NMR and ¹³C-NMR were obtained using Varian Unity Plus 400 MHz FT-NMR, a nuclear magnetic resonance spectrometer. CDCl₃ with 7.265 ppm of and CDCl₃ with 77.0 ppm of δ were used as internal standards to determine chemical shift. Coupling constant is referred to as J, and the unit is Hz.

Electrospray ionization mass spectra and electron impact mass spectra were obtained using API-3000™, available from Applied Biosystems, Inc.

### Preparation of Benzoxazinone Compounds of Formula (I)

The benzoxazinone compound of formula (I) according to the present invention was prepared based on the following scheme 1 or scheme 2. To be specific, for preparing a compound of 7-chloro-2-(2′-substituted-phenyl)-4H-benzo[d][1,3]oxazin-4-one, in the presence of pyridine, 2-amino-4-chlorobenzoic acid (compound a1) was reacted with benzoyl chloride substituted with R₃, in which R₃ is H, a halogen group, a C₁-C₄ alkyl group, or a C₁-C₄ alkoxyl group, at room temperature for 24 hours (see scheme 1).

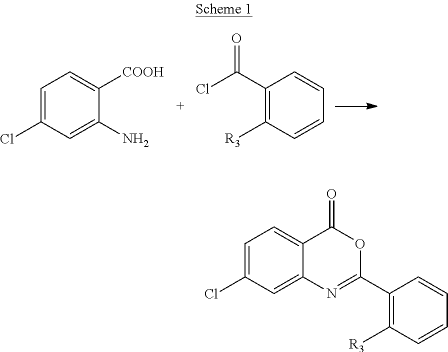

The compound of 5-chloro-2-(2′-substituted-phenyl)-4H-benzo[d][1,3]oxazin-4-one was prepared by reacting, in the presence of pyridine, 2-amino-6-chlorobenzoic acid (compound a2) with benzoyl chloride substituted with R₃, in which R₃ is H, a halogen group, a C₁-C₄ alkyl group, or a C₁-C₄ alkoxyl group, at room temperature for 24 hours (see scheme 2).

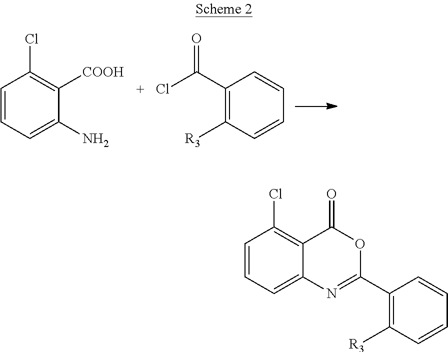

The benzoxazinone compounds of the present invention prepared using the aforesaid scheme 1 or scheme 2 are illustrated below.

### Example 1

### 7-chloro-2-(2′-fluorophenyl)-4H-benzo[d][1,3]oxazin-4-one (compound E1)

85 mg of 2-amino-4-chlorobenzoic acid commercially available from ACROS Organics, and 80 mg of 2-fluorobenzoyl chloride commercially available from ACROS Organics were added into 5 ml of pyridine. The mixture was stirred at room temperature for 24 hrs, followed by removal of the solvent at vacuum. The residue was purified using a silica gel column (CHCl₃/n-Hexane=1:3), thereby obtaining the title compound E1 of a white powder (117 mg, yield 85%).

### Structure Identification

The structure of the product thus obtained was identified using NMR and MASS. ¹H-NMR (400 MHz, CDCl₃): δ=8.16 (1H, d, J=8.0 Hz), 8.11 (1H, dt, J=2.0, 8.0 Hz), 7.70 (1H, d, J=2.0 Hz), 7.56 (1H, m), 7.50 (1H, dd, J=2.0, 8.0 Hz), 7.29 (1H, br.t, J=8.0 Hz), 7.22 (1H, dd, J=8.0, 8.0 Hz); ¹³C-NMR (100 MHz, CDCl₃): δ=161.5 (s, J_{C-F}=260.0 Hz), 158.3 (s), 147.7 (s), 143.0 (s), 134.4 (d, J_{C-F}=8.4 Hz), 131.2 (d), 131.2 (s), 129.8 (d), 129.2 (d), 127.2 (d), 124.3 (d), 124.3 (s), 117.3 (d, J_{C-F}=21.2 Hz), 115.3 (s); ESI-MS m/z 298 (100) [M+Na]⁺, 300 (31)

### Example 2

### 7-chloro-2-(2′-chlorophenyl)-4H-benzo[d][1,3]oxazin-4-one (compound E2)

The steps for preparing the title compound E2 in Example 2 were similar to those of Example 1. The differences reside in that the amount of 2-amino-4-chlorobenzoic acid was 82 mg, and 80 mg of 2-fluorobenzoyl chloride was replaced by 88 mg of 2-chlorobenzoyl chloride commerically available from ACROS Organics. A pale-yellow powder (108 mg) (i.e., the title compound E2) was obtained (yield 75%).

### Structure Identification

The structure of the product thus obtained was identified using NMR and MASS. ¹H-NMR (400 MHz, CDCl₃): δ=8.18 (1H, d, J=8.0 Hz), 7.90 (1H, dd, J=2.0, 8.0 Hz), 7.72 (1H, d, J=2.0 Hz), 7.53 (2H, m), 7.48 (1H, dd, J=2.0, 8.0 Hz), 7.40 (1H, dt, J=2.0, 8.0 Hz); ¹³C-NMR (100 MHz, CDCl₃): δ=158.4 (s), 157.7 (s), 147.5 (s), 143.1 (s), 133.6 (s), 132.6 (d), 131.5 (d), 131.2 (d), 129.8 (d), 129.8 (s), 129.5 (d), 127.2 (d), 126.9 (d), 115.3 (s); ESI-MS m/z 314 (100) [M+Na]⁺, 316 (61).

### Example 3

### 7-chloro-2-(2′-bromophenyl)-4H-benzo[d][1,3]oxazin-4-one (compound E3)

The steps for preparing the title compound E3 in Example 3 were similar to those of Example 1. The differences reside in that the amount of 2-amino-4-chlorobenzoic acid was 80 mg, and 80 mg of 2-fluorobenzoyl chloride was replaced by 120 mg of 2-bromobenzoyl chloride commerically available from ACROS Organics. A pale-yellow powder (113 mg) (i.e., the title compound E3) was obtained (yield 67%).

### Structure Identification

The structure of the product thus obtained was identified using NMR and MASS. ¹H-NMR (400 MHz, CDCl₃) δ=8.20 (1H, dd, J=3.0, 8.0 Hz), 7.86 (1H, br.d, J=8.0 Hz), 7.73 (1H, dd, J=2.0, 8.0 Hz), 7.73 (1H, br.s), 7.54 (1H, br.d, J=8.0 Hz), 7.46 (1H, t, J=8.0 Hz), 7.39 (1H, t, J=8.0 Hz); ¹³C-NMR (100 MHz, CDCl₃): δ=158.4 (s), 158.2 (s), 147.3 (s), 143.1 (s), 134.4 (d), 132.6 (d), 131.6 (s), 131.5 (d), 129.9 (d), 129.5 (d), 127.5 (d), 127.2 (d), 121.8 (s), 115.3 (s); ESI-MS m/z 360 (100) [M+Na]⁺, 358 (72).

### Example 4

### 7-chloro-2-(2′-methoxylphenyl)-4H-benzo[d][1,3]oxazin-4-one (compound E4)

The steps for preparing the title compound E4 in Example 4 were similar to those of Example 1. The differences reside in that the amount of 2-amino-4-chlorobenzoic acid was 79 mg, and 80 mg of 2-fluorobenzoyl chloride was replaced by 85 mg of 2-methoxybenzoyl chloride commerically available from ACROS Organics. The ratio of CHCl₃/n-Hexane was 2:5. 83 mg of a light yellow powder (i.e., the title compound E4) was obtained (yield 58%).

### Structure Identification

The structure of the product thus obtained was identified using NMR and MASS. ¹H-NMR (400 MHz, CDCl₃): δ=8.14 (1H, d, J=8.4 Hz), 7.85 (1H, dd, J=2.0, 8.4 Hz), 7.68 (1H, d, J=2.0 Hz), 7.50 (1H, dt, J=2.0, 8.4 Hz), 7.46 (1H, dd, J=2.0, 8.4 Hz), 7.05 (1H, t, J=8.4 Hz), 7.03 (1H, t, J=8.4 Hz), 3.92 (3H, s); ¹³C-NMR (100 MHz, CDCl₃): δ=159.0 (s), 158.8 (s), 158.7 (s), 148.0 (s), 142.6 (s), 133.5 (d), 131.4 (d), 129.6 (d), 128.8 (d), 126.9 (d), 120.5 (d), 119.9 (s), 115.2 (s), 112.1 (d), 56.0 (q); ESI-MS m/z 310 (100) [M+Na]⁺, 312 (30).

### Example 5

### 5-chloro-2-(2′-fluorophenyl)-4H-benzo[d][1,3]oxazin-4-one (compound E5)

The steps for preparing the title compound E5 in Example 5 were similar to those of Example 1. The differences reside in that 85 mg of 2-amino-4-chlorobenzoic acid was replaced by 81 mg of 2-amino-6-chlorobenzoic acid commerically available from ACROS Organics, and the amount of 2-fluorobenzoyl chloride was 85 mg. A light yellow powder (88 mg) (i.e., the title compound E5) was obtained (yield 640).

### Structure Identification

The structure of the product thus obtained was identified using NMR and MASS. ¹H-NMR (400 MHz, CDCl₃): δ=8.10 (1H, dt, J=2.0, 8.0 Hz), 7.69 (1H, t, J=8.0 Hz), 7.60 (1H, dd, J=0.4, 8.0 Hz), 7.55 (2H, m), 7.28 (1H, t, J=8.4 Hz), 7.21 (1H, dd, J=8.4, 8.0 Hz); ¹³C-NMR (100 MHz, CDCl₃): δ=161.4 (s, J_{C-F}=259.3 Hz), 155.6 (s), 148.9 (s), 135.9 (s), 134.3 (d, J_{C-F}=9.1 Hz), 131.7 (s), 131.1 (d, 2C), 126.4 (d), 126.0 (s), 124.3 (d, J_{C-F}=3.7 Hz), 121.2 (s), 117.3 (d, J_{C-F}=21.2 Hz), 114.7 (s); ESI-MS m/z 298 (100) [M+Na]⁺, 300 (34).

### Example 6

### 5-chloro-2-(2′-chlorophenyl)-4H-benzo[d][1,3]oxazin-4-one (compound E6)

The steps for preparing the title compound E6 in Example 6 were similar to those of Example 5. The differences reside in that the amount of 2-amino-6-chlorobenzoic acid was 79 mg, and 85 mg of 2-fluorobenzoyl chloride was replaced by 86 mg of 2-chorobenzoyl chloride. The ratio of CHCl₃/n-Hexane was 2:7. A light yellow powder (74 mg) (i.e., the title compound E6) was obtained (yield 51%).

### Structure Identification

The structure of the product thus obtained was identified using NMR and MASS. ¹H-NMR (400 MHz, CDCl₃): δ=7.90 (1H, dd, J=1.6, 8.0 Hz), 7.71 (1H, t, J=8.0 Hz), 7.61 (1H, dd, J=1.6, 8.0 Hz), 7.57 (1H, dd, J=1.6, 8.0 Hz), 7.51 (1H, dd, J=1.6, 8.0 Hz), 7.47 (1H, dt, J=1.6, 8.0 Hz), 7.40 (1H, dt, J=1.6, 8.0 Hz); ¹³C-NMR (100 MHz, CDCl₃): δ=157.2 (s), 155.8 (s), 148.7 (s), 136.0 (d), 133.5 (s), 132.5 (d), 131.4 (d), 131.3 (d), 131.3 (s), 131.2 (d), 129.7 (s), 126.9 (d), 126.4 (d), 114.7 (s); ESI-MS m/z 314 (100) [M+Na]⁺, 316 (64).

### Example 7

### 5-chloro-2-(2′-bromophenyl)-4H-benzo[d][1,3]oxazin-4-one (compound E7)

The steps for preparing the title compound E7 in Example 7 were similar to those of Example 5. The differences reside in that the amount of 2-amino-6-chlorobenzoic acid was 82 mg, and 85 mg of 2-fluorobenzoyl chloride was replaced by 124 mg of 2-brorobenzoyl chloride. The ratio of CHCl₃/n-Hexane was 1:4. A light yellow powder (52 mg) (i.e., the title compound E7) was obtained (yield 63%).

### Structure Identification

The structure of the product thus obtained was identified using NMR and MASS. ¹H-NMR (400 MHz, CDCl₃): δ=7.85 (1H, dd, J=1.6, 8.0 Hz), 7.72 (1H, dd, J=1.6, 8.0 Hz), 7.72 (1H, t, J=8.0 Hz), 7.62 (1H, dd, J=1.6, 8.0 Hz), 7.58 (1H, dd, J=1.6, 8.0 Hz), 7.45 (1H, dt, J=1.6, 8.0 Hz), 7.38 (1H, dt, J=1.6, 8.0 Hz); ¹³C-NMR (100 MHz, CDCl₃): δ=157.8 (s), 155.8 (s), 148.6 (s), 136.0 (d), 134.4 (d), 132.6 (d), 131.7 (s), 131.4 (d), 131.4 (d), 131.3 (s), 127.5 (s), 126.4 (d), 121.8 (s), 114.7 (s); ESI-MS m/z 360 (100) [M+Na]⁺, 358 (69).

### Example 8

### 5-chloro-2-(2′-methylphenyl)-4H-benzo[d][1,3]oxazin-4-one (compound E8)

The steps for preparing the title compound E8 in Example 8 were similar to those of Example 5. The differences reside in that the amount of 2-amino-6-chlorobenzoic acid was 77 mg, and 85 mg of 2-fluorobenzoyl chloride was replaced by 84 mg of 2-methylbenzoyl chloride commerically available from ACROS Organics. The ratio of CHCl₃/n-Hexane was 1:4. Alight yellow powder (81 mg) (i.e., the title compound E8) was obtained (yield 60%).

### Structure Identification

The structure of the product thus obtained was identified using NMR and MASS. ¹H-NMR (400 MHz, CDCl₃): δ=8.03 (1H, br.d, J=8.0 Hz), 7.68 (1H, t, J=8.0 Hz), 7.58 (1H, dd, J=1.2, 8.0 Hz), 7.52 (1H, dd, J=1.2, 8.0 Hz), 7.43 (1H, dt, J=1.2, 8.0 Hz), 7.32 (2H, br.t, J=8.0 Hz), 2.72 (3H, s); ¹³C-NMR (100 MHz, CDCl₃): δ=158.9 (s), 156.2 (s), 149.1 (s), 139.4 (s), 135.8 (d), 132.0 (d), 131.9 (d), 131.9 (s), 130.7 (d), 130.2 (d), 129.1 (s), 126.2 (d), 126.1 (d), 114.5 (s) 22.2 (q); ESI-MS m/z 294 (100) [M+Na]⁺, 292 (31).

### Example 9

### 5-chloro-2-(2′-methoxylphenyl)-4H-benzo[d][1,3]oxazin-4-one (compound E9)

The steps for preparing the title compound 59 in Example 9 were similar to those of Example 5. The differences reside in that the amount of 2-amino-6-chlorobenzoic acid was 80 mg, and 85 mg of 2-fluorobenzoyl chloride was replaced by mg of 2-methoxybenzoyl chloride. The ratio of CHCl₃/n-Hexane was 2:7. A light yellow powder (70 mg) (i.e., the title compound E9) was obtained (yield 49%).

### Structure Identification

The structure of the product thus obtained was identified using NMR and MASS. ¹H-NMR (400 MHz, CDCl₃) δ=7.85 (1H, dd, J=1.6, 8.0 Hz), 7.67 (1H, t, J=8.0 Hz), 7.59 (1H, br.dd, J=1.6, 8.0 Hz), 7.51 (1H, dd, J=1.6, 8.0 Hz), 7.50 (1H, dt, J=1.6, 8.0 Hz), 7.06 (1H, br.t, J=8.0 Hz), 7.03 (1H, br.t, J=8.0 Hz), 3.92 (3H, s); ¹³C-NMR (100 MHz, CDCl₃): δ=158.7 (s), 156.4 (s), 149.3 (s), 135.7 (d), 133.5 (d), 132.6 (s), 131.3 (d), 130.7 (d), 126.2 (d), 125.1 (s), 121.3 (s), 120.5 (d), 114.6 (s), 112.1 (d), 56.0 (q); ESI-MS m/z 310 (100) [M+Na]⁺, 312 (32).

### Comparative Example 1

### 8-chloro-2-(2′-fluorophenyl)-4H-benzo[d][1,3]oxazin-4-one (compound CE1)

8-chloro-2-(2′-fluorophenyl)-4H-benzo[d][1,3]oxazin-4-one was prepared based on the method disclosed in P. W. Hsieh et al., Bioorg. Med. Chem. Lett., 15:2786-2789, 2005. In brief, 117 mg of 2-amino-3-chlorobenzoic acid commerically available from ACROS Organics and 177 mg of 2-fluorobenzoyl chloride commerically available from ACROS Organics were added into 5 ml of pyridine. The mixture was stirred at room temperature for 16 hrs, followed by removal of the solvent at vacuum. The residue was purified using a silica gel column (CHCl₃/n-Hexane=1:3), thereby obtaining the title compound (i.e., compound CE1) of a pale-yellow powder (184 mg, yield 67%).

### Structure Identification

The structure of the product thus obtained was identified using NMR and MASS. ¹H-NMR (400 MHz, CDCl₃): δ=8.19 (1H, t, J=8.0, 0.8 Hz, Ar—H), 8.15 (1H, d, J=8.0 Hz, Ar—H), 7.89 (1H, d, J=8.0 Hz, Ar—H), 7.56 (1H, m, Ar—H), 7.46 (1H, t, J=8.0 Hz, Ar—H), 7.29 (1H, t, J=8.0 Hz, Ar—H), 7.22 (1H, dd, J=8.0, 1.2 Hz, Ar—H); EI-MS m/z: 275 [M]⁺ (91), 277 (32).

### Comparative Example 2

### 8-chloro-2-(2′-chlorophenyl)-4H-benzo[d][1,3]oxazin-4-one (compound CE2)

The steps for preparing the title compound CE2 in Comparative Example 2 were similar to those of Comparative Example 1. The differences reside in that the amount of 2-amino-3-chlorobenzoic acid was 120 mg, and 177 mg of 2-fluorobenzoyl chloride was replaced by 200 mg of 2-chlorobenzoyl chloride. 180 mg of a pale-yellow powder (i.e., the title compound CE2) was obtained (yield 630).

### Structure Identification

The structure of the product thus obtained was identified using NMR and MASS. ¹H-NMR (400 MHz, CDCl₃): δ=8.17 (1H, dd, J=8.0, 1.6 Hz, Ar—H), 8.01 (1H, dd, J=8.0, 1.6 Hz, Ar—H), 7.91 (1H, dd, J=8.0, 1.2 Hz, Ar—H), 7.54 (1H, dd, J=8.0, 1.2 Hz, Ar—H), 7.49 (1H, t, J=8.0 Hz, Ar—H), 7.47 (1H, dd, J=8.0, 1.6 Hz, Ar—H), 7.41 (1H, td, J=8.0, 1.2 Hz, Ar—H); EI-MS m/z: 291 [M]⁺ (89), 293 (56).

### Comparative Example 3

### 8-chloro-2-(2′-bromophenyl)-4H-benzo[d][1,3]oxazin-4-one (compound CE3)

The steps for preparing the title compound CE3 in Comparative Example 3 were similar to those of Comparative Example 1. The differences reside in that the amount of 2-amino-3-chlorobenzoic acid was 120 mg, and 177 mg of 2-fluorobenzoyl chloride was replaced by 224 mg of 2-bromobenzoyl chloride. 165 mg of a light yellow powder (i.e., the title compound CE3) was obtained (yield 50%).

### Structure Identification

The structure of the product thus obtained was identified using NMR and MASS. ¹H-NMR (400 MHz, CDCl₃) δ=8.18 (1H, dd, J=8.0, 1.6 Hz, Ar—H), 7.97 (1H, dd, J=8.0, 1.6 Hz, Ar—H), 7.91 (1H, dd, J=8.0, 1.6 Hz, Ar—H), 7.75 (1H, dd, J=8.0, 1.2 Hz, Ar—H), 7.50 (1H, t, J=8.0 Hz, Ar—H), 7.45 (1H, dd, J=8.0, 1.2 Hz, Ar—H), 7.39 (1H, td, J=8.0, 1.6 Hz, Ar—H); EI-MS m/z: 337 [M]⁺ (98), 335 (78).

### Comparative Example 4

### 8-chloro-2-(2′-methylphenyl)-4H-benzo[d][1,3]oxazin-4-one (compound CE4)

The steps for preparing the title compound CE4 in Comparative Example 4 were similar to those of Comparative Example 1. The differences reside in that the amount of 2-amino-3-chlorobenzoic acid was 175 mg, and 177 mg of 2-fluorobenzoyl chloride was replaced by 214 mg of 2-methylbenzoyl chloride. 110 mg of a light yellow powder (i.e., the title compound CE4) was obtained (yield 41%).

### Structure Identification

The structure of the product thus obtained was identified using NMR and MASS. ¹H-NMR (400 MHz, CDCl₃) δ=8.13 (2H, dd, J=8.0, 1.2 Hz, Ar—H), 7.86 (1H, dd, J=8.0, 0.8 Hz, Ar—H), 7.42 (1H, m, Ar—H), 7.42 (1H, t, J=8.0 Hz, Ar—H), 7.32 (2H, m, Ar—H), 2.82 (3H, s, Me); EI-MS m/z: 271 [M]⁺ (100), 273 (33).

### Comparative Example 5

### 8-chloro-2-(2′-methoxylphenyl)-4H-benzo[d][1,3]oxazin-4-one (compound CE5)

The steps for preparing the title compound CE5 in Comparative Example 5 were similar to those of Comparative Example 1. The differences reside in that the amount of 2-amino-3-chlorobenzoic acid was 167 mg, and 177 mg of 2-fluorobenzoyl chloride was replaced by 220 mg of 2-methoxybenzoyl chloride. 146 mg of a light yellow powder (i.e., the title compound CE5) was obtained (yield 51%).

### Structure Identification

The structure of the product thus obtained was identified using NMR and MASS. ¹H-NMR (400 MHz, CDCl₃) δ=8.15 (1H, dd, J=8.0, 1.0 Hz, Ar—H), 7.98 (1H, dd, J=8.0, 1.6 Hz, Ar—H), 7.86 (1H, dd, J=8.0, 1.0 Hz, Ar—H), 7.51 (1H, td, J=8.0, 1.2 Hz, Ar—H), 7.43 (1H, t, J=8.0 Hz, Ar—H), 7.07 (1H, t, J=8.0 Hz, Ar—H), 7.03 (1H, d, J=8.0 Hz, Ar—H), 3.95 (3H, s, OMe); EI-MS m/z: 287 [M]⁺ (29), 289 (14).

### Pharmacological Experiments of the Benzoxazinone Compounds E1 to E9

### Material

1. Ca²⁺-free Hank's balanced salt solution having pH 7.4 includes ingredients shown in Table 1, the ingredients were dissolved in deionized water.

TABLE 1

| Ingredient | Conc. (mg/L) |
|---|---|
| NaCl | 8000 |
| KCl | 400 |
| KH₂PO₄ | 60 |
| Glucose | 1000 |
| Na₂HPO₄ | 48 |
| MgCl₂•6H₂O | 203 |
| NaHCO₃ | 350 |

2. Hank's balanced salt solution (HBSS) having pH 7.4 includes ingredients shown in Table 2, the ingredients were dissolved in deionized water.

TABLE 2

| Ingredient | Conc. (mg/L) |
|---|---|
| CaCl₂•2H₂O | 294 |
| NaCl | 8000 |
| KCl | 400 |
| KH₂PO₄ | 60 |
| Glucose | 1000 |
| Na₂HPO₄ | 48 |
| MgCl₂•6H₂O | 203 |
| NaHCO₃ | 350 |

3. Human neutrophils

Human neutrophils were prepared by the following procedure. Blood obtained from healthy human donors (20-32 years old) by venipuncture was centrifuged at 650 g for 10 minutes, and a leukocyte-rich lower layer was obtained by removing the upper layer containing platelets. 3% dextran T500 solution was well mixed with the leukocyte-rich lower layer at a volume ratio of 1:1 and was allowed to stand at room temperature. After standing for 30 minutes, the clear supernatant containing neutrophils was slowly transferred to a centrifuge tube of Ficoll-Pague™ Plus (14-1440-03, GE Healthcare, Sweden) and was subjected to density-gradient centrifugation at 400 g and 20° C. for 40 minutes. The precipitate thus obtained was treated with a hypotonic solution, i.e., 0.2% NaCl, to lyse erythrocytes, followed by removal of the lysed erythrocytes using centrifugation at 200 g and 4° C. for 8 minutes so as to obtain the neutrophils. The purified neutrophils having >98% viable cells determined using a trypan blue exclusion method was suspended in Ca²⁺-free Hank's balanced salt solution (pH 7.4), thereby obtaining a neutrophil suspension having a concentration of 1.2×10⁶ cell/ml. The neutrophil suspension was stored at 4° C. before use.

### Pharmacological Experiments:

### A. Effect of the Benzoxazinone Compounds E1 to E9 and CE1 to CE5 on Generation of Superoxide Anion O₂

The O₂^{•-} generation assay was based on the superoxide dismutase (SCD)-inhinitable reduction of ferricytochrome c. In brief, each of the compounds of E1-E9 and CE1-CE5 was dissolved in 100% DMSO so as to obtain a compound solution. The neutrophils obtained in the section of “3. Human neutrophils” were added with ferricytochrome c (dissolved in HBSS at a concentration of 1.0 mg/ml) at a volume ratio of 1:1 (the final volume was 1.5 ml), stirred at 37° C. for 2 minutes, followed by addition with 1.5 μl of the aforesaid compound solution and reaction at 37° C. for 2 minutes. In a control group, 100% DMSO was used to substitute the compound solution. Moreover, in the positive control group, diphenyleneiodonium (DPI) was used to substitute the compound solution. The reacted mixture was added with 1.5 μl of cytochalasin B (CB, 1 mg/ml) and was incubated for minutes, followed by addition with 1.5 μl of formyl-L-methionyl-L-leucyl-L-phenylalanine (FMLP, 100 μM) and reaction for 10 minutes to activate neutrophils. Change in absorbance for the final mixture at 550 nm was measured using a spectrophotometer (U-3010, Hitachi). IC₅₀ value, indicating the concentration of the benzoxazinone compound that reduces 50% of absorbance of the final mixture as compared with the control group, was recorded. The results are shown in Table 3.

### B. Effect of the Benzoxazinone Compounds E1 to E9 and CE1 to CE5 on Release of Human Neutrophil Elastase (NE)

Each of the compounds of E1-E9 and CE1-CE5 was dissolved in 100% DMSO so as to obtain a compound solution. The neutrophils obtained in the section of “3. Human neutrophils” was added with 200 μM of MeO-Suc-Ala-Ala-Pro-Val-p-nitroanilide (454454, Calbiochem, dissolved in HBSS and functioned as a substrate for human neutrophils) at a volume ratio of 1:1 (the final volume was 1.5 ml), stirred at 37° C. for 2 minutes, followed by addition with 1.5 μl of the aforesaid compound solution and reaction at 37° C. for 2 minutes. In a control group, 100% DMSO was used to substitute the compound solution. Moreover, phenylmethylsulfonyl fluoride (PMSF) and elastatinal (PI-103, Enzo Life Science, USA) were used to substitute the compound solution as positive control groups. The reacted mixture was added with 1.5 μl of cytochalasin B (CB, 0.5 mg/ml) and was incubated for 3 minutes, followed by addition with 1.5 μl of formyl-L-methionyl-L-leucyl-L-phenylalanine (FMLP, 100 μM) and reaction for 10 minutes to activate neutrophils. Change in absorbance for the final mixture at 405 nm was measured using a spectrophotometer (U-3010, Hitachi). IC₅₀ value, indicating the concentration of the benzoxazinone compound that reduces 50% of absorbance of the final mixture as compared with the control group, was recorded. The results are shown in Table 3.

TABLE 3

IC₅₀ (nM)^{a}

| Compound | O₂^{•-} generation | NE release |
|---|---|---|
| DPI | 969.7 ± 394.6 | Not tested |
| PMSF | Not tested | 109518.5 ± 25039.7 |
| Elastatinal | Not tested | 56450.0 ± 9130.0 |
| CE1 | >25000 | 6030.0 ± 650.0 |
| CE2 | 8660.0 ± 1440.0 | 1890.0 ± 1100.0 |
| CE3 | 6560.0 ± 3090.0 | 1690.0 ± 300.0 |
| CE4 | 17490.0 ± 2690.0 | >25000 |
| CE5 | 10030.0 ± 590.0 | 1910.0 ± 140.0 |
| E1 | 1381.8 ± 129.6 | 1141.0 ± 144.4 |
| E2 | 2680.5 ± 90.8 | 836.8 ± 68.4 |
| E3 | 2002.1 ± 158.5 | 536.3 ± 91.8 |
| E4 | 1206.1 ± 127.1 | 227.5 ± 47.4 |
| E5 | 23393.8 ± 1232.9 | 77.1 ± 15.3 |
| E6 | >25000 | 47.1 ± 9.6 |
| E7 | >25000 | 80.8 ± 10.3 |
| E8 | >25000 | 47.6 ± 4.3 |
| E9 | 6475.5 ± 1408.0 | 64.4 ± 12.9 |

^{a}IC₅₀ values are presented by mean ± S.E.M. (n = 3)

It is apparent from Table 3 that the benzoxazinone compounds E1 to E4 of the present invention exhibit good inhibition of O₂^{•-} generation. The benzoxazinone compounds E5 to E9 of the present invention have good inhibition of NE release. The results suggest that the benzoxazinone compounds of the present invention have superior anti-inflammatory activity.

### C. Effect of the Benzoxazinone Compounds E4 to E9 on Inhibition of NE Activity

In this experiment, the benzoxazinone compounds E4 to E9 of the present invention were used to investigate the inhibition effect.

Each of the benzoxazinone compounds E4 to E9 was dissolved in 100% DMSO so as to obtain a compound solution. The neutrophils obtained in the section of “3. Human neutrophils” were added into HESS at a volume ratio of 1:1 (the final volume was 1.5 ml), stirred at 37° C. for 2 minutes, followed by addition with 1.5 μl of cytochalasin B (CB, 1.5 mg/ml) and incubation for 3 minutes. After incubation, the mixture was added with 1.5 μl of FMLP (100 μM) and was reacted for 20 minutes to activate neutrophils, followed by centrifugation at 1000 g and 4° C. for 5 minutes, thereby obtaining a supernatant containing human neutrophil elastase. The supernatant was stored at 4° C. before use. 1.5 ml of the supernatant containing human neutrophil elastase was stirred at 37° C. for 2 minutes and was added with 1.5 μl of the compound solution, followed by reaction for 5 minutes at 37° C. In a control group, 100% DMSO was used to substitute the compound solution. Moreover, in the positive control group, PMSF was used to substitute the compound solution. 1.5 μl of MeO-Suc-Ala-Ala-Pro-Val-p-nitroanilide (100 mM) was added into the reacted mixture and was reacted for 10 minutes so as to obtain a reaction product. Absorbance of the reaction product was measured at 405 nm using an ELISA reader. IC₅₀ value, indicating the concentration of the benzoxazinone compound that reduces 50% of absorbance of the reaction product as compared with the control group, was recorded. The results are shown in Table 4.

TABLE 4

| Compound | IC₅₀ (nM)^{a} |
|---|---|
| PMSF | 95219.30 ± 19225.81 |
| E4 | 612.39 ± 179.79 |
| E5 | 52.72 ± 4.50 |
| E6 | 30.74 ± 3.96 |
| E7 | 24.77 ± 1.89 |
| E8 | 57.06 ± 9.23 |
| E9 | 40.04 ± 4.63 |

^{a}IC₅₀ values are presented by mean ± S.E.M. (n = 3)

The effect of each of the benzoxazinone compounds E4 to E9 on inhibition of commercially available pure neutrophil elastase (NE) was also investigated.

Each of the benzoxazinone compounds E4 to E9 was dissolved in 100% DMSO and was diluted with Ca²⁺-free Hank's balanced salt solution (1:1000) so as to obtain a compound solution. 100 μl of HESS buffer having pH of 7.4 and containing 0.0075 U/ml human NE (EC 3.421.37 Sigma, St. Louis, Mo. USA) was added into a 96-well microtiter plate and subsequently was added with 50 μl of the compound solution, followed by addition with 100 μl of MeO-Suc-Ala-Ala-Pro-Val-p-nitroanilide (0.05 μmol) and reaction for 30 minutes. In a control group, 0.1% DMSO was used to substitute the compound solution. Moreover, in the positive control group, PMSF was used to substitute the compound solution. Absorbance of the reaction product was measured at 405 nm using an ELISA reader (Multiskan Ascent, Thermo). IC₅₀ value, indicating the concentration of the benzoxazinone compound that reduces 50% of absorbance of the reaction product as compared with the control group, was recorded. The results are shown in Table 5.

TABLE 5

| Compound | IC₅₀ (nM)^{a} |
|---|---|
| PMSF | 52161.7 ± 1073.4 |
| 4 | 221.49 ± 59.20 |
| 5 | 30.77 ± 5.82 |
| 6 | 28.74 ± 6.16 |
| 7 | 29.51 ± 6.71 |
| 8 | 48.83 ± 14.17 |
| 9 | 15.37 ± 1.91 |

As shown in Tables 4 and 5, compared with PMSF (i.e., the positive control group), the benzoxazinone compounds E4 to E9, especially compounds E5 to E9, more especially compound E7, exhibit superior effect on inhibition of NE activity. The results suggest possible use of the benzoxazinone compounds of the present invention as an anti-inflammatory drug.

### D. Effect of the Benzoxazinone Compound E7 on Treatment of Lung Injury Caused by Trauma-Hemorrhagic Shock

To further prove the effect of the benzoxazinone compound E7 on treatment of inflammatory disorders, benzoxazinone compound E7 was further subjected to an animal test. Specifically, the effects of compound E7 on water content in lung tissues and myeloperoxidase (MPO) activity were measured.

I. Induction of Trauma-Hemorrhagic Shock

The procedure for induction of trauma-hemorrhagic shock was based on the method disclosed in H. P. Yu et al., Surgery, 138:85-92, 2005 and H. P. Yu et al., Biochem. Pharmacol., 78:983-992, 2009. In brief, male Sprague-Dawley (S.D.) rats having a body weight ranging from 275 to 325 g were obtained from National Laboratory Animal Center. The rats were randomly divided into four groups including experimental groups 1 and 2 and sham-operated groups 1 and 2. Each group included 8 rats. All rats were raised in an air-conditioned room with the following conditions: a light-dark cycle of 12-14 hours of illumination and 10-12 hours of darkness, 20-26° C., and relative humidity of 40-60%. The rats were free to have sufficient drinking water and feed and lived in the aforesaid room for one week to adapt to the environment. Before beginning the experiments, the rats were starved overnight while allowed to have sufficient drinking water ad libitum. The rats were anesthetized by isoflurane (Attane, Minrad, Bethlehem, Pa.) inhalation and were subjected to 5-cm midline laparotomy in the abdomen. Three catheters, i.e., PE-50 polyethylene tubing, commercially available from Becton Dickinson, Sparks, Md., were inserted into the left femoral artery, the right femoral artery, and the right femoral vein of each rat. The abdomen was sutured. During the surgical procedure, wounds were bathed in 1% lidocaine commercially available from Elkins-Sinn, Cherry Hill, N.J., to reduce postoperative pain. The rats were then allowed to waken and to be bled, and the mean blood pressure was maintained at 40 mmHg. The level of hypotension was maintained until the rat could not maintain a mean blood pressure at 40 mmHg unless additional fluid of Ringer's lactate was administrated (referred to as maximum bleed-out). The total amount of the blood volume of maximum bleed-out was recorded. The rats were then maintained at a mean blood pressure of 40 mmHg by administrating 40% of the blood volume of maximumbleed-out of the fluid of Ringer's lactate into each of the rats through the catheter inserted in the right femoral vein. The rat was gradually resuscitated by importing four times of the blood volume of maximum bleed-out of the fluid of Ringer's lactate into the rat for 60 minutes. The time required for arriving maximum bleed-out was about 45 minutes, the volume of maximum bleed-out was 60% of the circulating blood volume, and the time for hemorrhage was about 90 minutes. Thirty minutes before the end of the resuscitation period, the rats in experimental groups 1 and 2 were injected with an equal volume of the vehicle, i.e., 0.2 ml of 10% DMSO, and the compound E7, respectively. The catheters were then removed, the vessels were ligated, and the skin incisions were sutured. The sham-operated groups 1 and 2 were subjected to the aforesaid procedure except for the hemorrhage and resuscitation. An equal volume of the vehicle, i.e., 0.2 ml of 10% DMSO, and the compound E7 were injected into the sham-operated groups 1 and 2, respectively. The rats continued to live in the aforesaid room and were allowed to get the food and drinking water ad libitum. The rats were sacrificed after 24 hours of the end of resuscitation.

II. Preparation of Lung Tissue

The chest of each of the rats was opened and the left side of the lung was obtained by clamping the hilum. Excess blood was removed and the left upper lobe of the lung was used for water content assay in the following section III. Other tissue sections were used for myeloperoxidase activity assay in the following section IV.

III. Water Content Assay

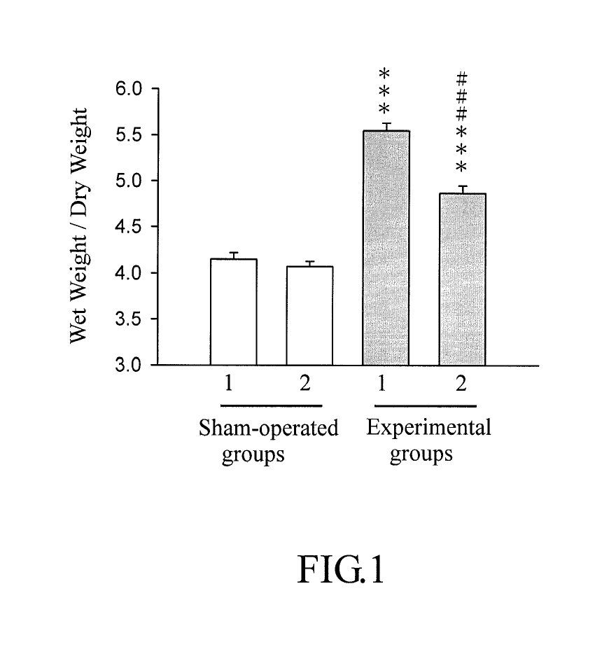

The left upper lobe was weighted and the weight was referred to as “wet weight”. The left upper lobe was then dried at 80° C. for 24 hours, and the weight, referred to as dry weight, thereof was measured. The water content of lung tissue was expressed by the ratio of wet weight/dry weight. The result is shown in FIG. 1.

IV. Analysis of Myeloperoxidase (MPO) Activity

Activity of MPO was determined based on a method disclosed in H. P. Yu et al., Biochem. Pharmacol., 78: 983-992, 2009. In brief, the lung tissues excluding the left upper lobe obtained from the rats in each group were suspended in 1 ml of buffer solution (0.5% hexadecyltrimethylammonium bromide in a 50 mM phosphate buffer, pH 6.0). The total weight of the lung tissues to be tested in each group was the same. The lung tissues were subjected to sonication on ice for 30 cycles two times, each cycle being conducted seconds, between the first and second times, the suspension was allowed to stand for 30 seconds. The suspension was subsequently subjected to centrifugation at 2000 g and 4° C. Total protein concentration (mg/ml) in the supernatant was determined using a Bio-Rad assay kit (Bio-Rad, Hercules, Calif.). 290 μL of phosphate buffer (50 mM), 3 μL of o-dianisidine hydrochloride solution (20 g/L, as a substrate for MPO), and 3 μL of H₂O₂ (20 mM) was added into each well of a 96-well plate. 10 μL of the supernatant was added into each well to start reaction for 3 to 5 minutes. The reaction was stopped using 3 μL of 30% sodium azide. Absorbance (OD₄₆₀) for the reaction mixture was measured at 460 nm using an ELISA Reader (Multiskan Ascent, Thermo). Absorbance (OD₄₆₀) was converted to MPO concentration based on a standard curve. The standard curve was obtained by plotting MPO standard commercially available from Sigma, St. Louis, Mo. at concentrations of 5 U/mL, 2.5 U/mL, 1.25 U/mL, 0.625 U/mL, 0.3125 U/mL, 0.15625 U/mL, and 0.078125 U/mL with the respective OD₄₆₀. The activity was determined based on the following equation:

MPO Activity (U/mg)=MPO concentration (U/ml)/total protein concentration (mg/ml)

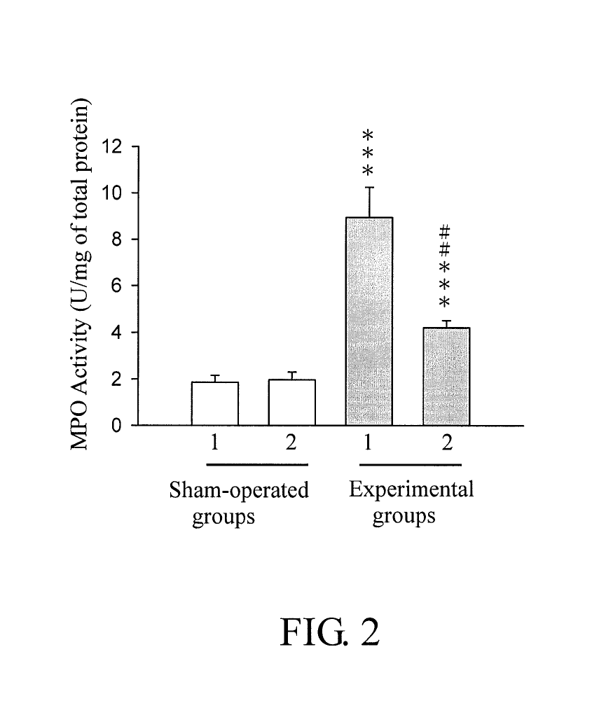

The data were analyzed using the GraphPad Prism software (GraphPad Software, San Diego, Calif.). Statistical analysis was performed using Student's t-test or one-way analysis of variance (ANOVA), followed by Turkey's multiple-comparison test. A value of p<0.05 was considered statistically significant. The result is shown in FIG. 2.

As shown in FIGS. 1 and 2, there is no significant difference in water content and MPO activity between the sham-operated groups 1 and 2. However, compared with the sham-operated groups, the water content and MPO activity in experimental groups were higher attributed to the trauma-hemorrhagic shock. Moreover, compared with experimental group 1, in which the rats were administrated with vehicle, administration of compound E7 to the rats in experimental group 2 significantly reduced water content and MPO activity. The data indicated that the compounds of the present invention can be used to treat lung injury induced by hemorrhagic shock.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. A benzoxazinone compound having the following formula (I): 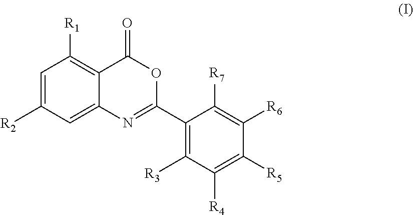
wherein
one of R₁ and R₂ is H, and the other of R₁ and R₂ is a halogen group, and R₁ and R₂ cannot be the same at the same time; and
R₃, R₄, R₅, R₆, and R₇ are independently selected from the group consisting of H, a halogen group, a C₁-C₄ alkyl group, and a C₁-C₄ alkoxyl group.

2. The benzoxazinone compound of claim 1, wherein one of R₁ and R₂ is H, and the other of R₁ and R₂ is Cl, and R₃, R₄, R₅, R₆, and R₇ are independently selected from the group consisting of H, F, Cl, Br, methyl, and methoxy.

3. The benzoxazinone compound of claim 2, wherein R₁ is H, R₂ is C₁, R₃ is selected from the group consisting of F, Cl, Br, and methoxy, and R₄, R₅, R₆, and R₇ are H.

4. The benzoxazinone compound of claim 2, wherein R₁ is C₁, R₂ is H, R₃ is selected from the group consisting of F, Cl, Br, methyl, and methoxy, and R₄, R₅, R₆, and R₇ are H.

5. The benzoxazinone compound of claim 2, selected from the group consisting of:
7-chloro-2-(2′-fluorophenyl)-4H-benzo[d][1,3]oxazin-4-one,
7-chloro-2-(2′-chlorophenyl)-4H-benzo[d][1,3]oxazin-4-one,
7-chloro-2-(2′-bromophenyl)-4H-benzo[d][1,3]oxazin-4-one,
7-chloro-2-(2′-methoxylphenyl)-4H-benzo[d][1,3]oxazin-4-one,
5-chloro-2-(2′-fluorophenyl)-4H-benzo[d][1,3]oxazin-4-one,
5-chloro-2-(2′-chlorophenyl)-4H-benzo[d][1,3]oxazin-4-one,
5-chloro-2-(2′-bromophenyl)-4H-benzo[d][1,3]oxazin-4-one,
5-chloro-2-(2′-methylphenyl)-4H-benzo[d][1,3]oxazin-4-one, and
5-chloro-2-(2′-methoxylphenyl)-4H-benzo[d][1,3]oxazin-4-one.

6. A pharmaceutical composition for treatment of an inflammatory disorder, comprising the benzoxazinone compound of claim 1 or the pharmaceutically acceptable salt thereof.

7. The pharmaceutical composition of claim 6, wherein the inflammatory disorder is selected from the group consisting of: lung injury, chronic obstructive pulmonary disease, acute respiratory distress syndrome, cystic fibrosis, ischemic-reperfusion injury, glomerulonephritis, arthritis, bullous pemphigoid, and sepsis.

8. A method for producing the benzoxazinone compound of claim 1, comprising:
reacting a compound of formula (II): 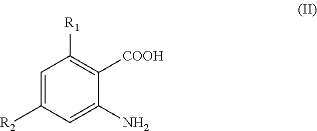
with a compound of formula (III): 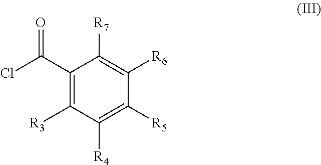
wherein R₁, R₂, R₃, R₄, R₅, R₆, and R₇ have the same definitions as R₁, R₂, R₃, R₄, R₅, R₆, and R₇ in claim 1, respectively.
